(19) United States
(12) Patent Application Publication
(10) Pub. No.: US 2002/0159980 A1
(43) Pub. Date: Oct. 31, 2002

(54) Benomyl tolerant fusarium lateritium and uses thereof

(76) Inventors: David E. Block, Davis, CA (US); Jean S. Vandergheynst, Davis, CA (US)

Correspondence Address: Rae-Venter Law Group, P.C., P.O. BOX 1898, MONTEREY, CA, 93942-1898, US

(21) Appl. No.: 09/796,635

(22) Filed: Feb. 28, 2001

(51) Int. Cl. A01N 63/04; C12N 1/14; C12N 15/01

(52) U.S. Cl. 424/93.5; 435/254.7; 435/441

(57) ABSTRACT

The invention provides methods for treating a plant having an exposed wound to increase resistance against a plant pathogen, by applying an effective amount of a benomyl solution, in conjunction with inoculating the pruning wound with a fungal organism resistant to the benomyl concentration. The invention also provides a benomyl tolerant F. lateritium which is particularly adapted for the treatment of grapevine pruning wounds to control Eutypa dieback.

### TECHNICAL FIELD

The present invention relates to compositions and methods for the biological control of plant diseases. More particularly, this invention relates to methods and compositions for controlling plant diseases using biological control agents.

### BACKGROUND OF THE INVENTION

Plant diseases, caused primarily by fungal and bacterial pathogens, cause severe losses to agricultural and horticultural crops every year. These losses can result in reduced food supplies, higher prices, poorer quality agricultural products, and economic hardship for growers and processors.

For many diseases, traditional chemical control methods are not always economical or effective, and some chemical controls may have unwanted health, safety, and environmental risks. Biological control involves the use of beneficial microorganisms, such as specialized fungi and bacteria, to attack and control plant pathogens and the diseases they cause. Biological control offers a safer and more environmentally healthy approach to the management of plant disease and can be incorporated with cultural and physical controls and limited chemical usage for an effective integrated disease management system. Biological control can be an important component in the development of more sustainable agriculture systems.

Advantages of using biological control in conjunction with an integrated pest or disease management approach is the replacement for more toxic agents with safer agents, the use of less of the expensive chemical controls, and fewer applications of agents of any kind, with the concomitant savings in time and money.

Although there has been considerable progress in the methods and techniques involved in the development of biological control agents (BCAs), the field has suffered a slow transition to wide-scale field application (U.S. Congress, Office of Technology Assessment. 1995. Biologically Based Technologies for Pest Control. Washington, D.C, U.S. Government Printing Office). One significant reason for the delay in the adoption of broad-scale biocontrol is that potential end-users doubt the efficacy of such measures as opposed to more traditional chemical control measures. Furthermore, issues associated with the production and delivery of BCAs have also limited their adoption.

There are numerous fungal plant diseases, which cause a variety of different types of symptoms. Examples include foliar pathogens (necrotrophs and biotrophs) and various soil-borne wilt diseases. Canker fungal diseases produce cankers that affect many varieties of trees and shrubs. The cankers will appear in several forms such as splits, sunken, or swollen areas of the plant and abnormal tissue growth. Once established the infected portion of the plant is typically pruned away, and for many canker diseases this is the only reliable treatment.

Eutypa lata is a very opportunistic and highly destructive pandemic fungal phytopathogen that causes the disease Eutypa Dieback in grapevine. In California it is estimated that as much as 20% of the total vineyard acreage (750,000 acres) is infected to some extent by E. lata. This disease occurs when the pruning wounds of grapevines become infected with spores from E. lata. If not prevented, infection can be lethal and replanting becomes the only course of action.

Over the last thirty years, benomyl, a broad-spectrum fungicide, has been the sole means of control of E. lata. Benomyl application is somewhat effective for short-term control of the disease. However, more long-term strategies for E. lata control are needed since wounds are susceptible for as long as four weeks after pruning and no post-infection eradication method currently exists. Additionally, application of benomyl in the field is often a laborious process, and would require multiple applications over a short period of time to achieve effective control. This type of multiple application is almost never economically feasible. Furthermore, tolerance of fungi to low levels of benomyl (0-10 μg/ml) has been reported in the literature, and the gene conferring this tolerance has been identified in several fungi (Orbach, et al., 1986; Gold, et al., 1991; Koenraadt, et al., 1992; and Yan, et al., 1996). Clearly, given the aforementioned reasons, a more effective means of control of E. lata is needed.

BCAs have been developed which are resistant to benzimadole fungicides. Ossana, et al. (1990) generated a benomyl resistance strain of Gliocladium virens through genetic engineering. Various United States patents claim strains of Trichoderma viride which are resistant to benomyl. (U.S. Pat. No. 4,915,944, assigned to Yissum Research Development Corp. of the Hebrew University of Jerusalem, U.S. Pat. No. 4,489,161, assigned to The United States of America as represented by the Secretary of Agriculture; and U.S. Pat. No. 5,418,165, assigned to The University of Alaska-Fairbanks). Both Gliocladium virens and Trichoderma viride are soil-borne fungi, and as BCA's are applied to soil and for seed treatments to control the growth of various soil pathogenic fungi, such as wilt diseases.

Application of BCAs that colonize the pruning wounds might offer more long-term control of various canker and die-back diseases, either by themselves or in combination with a fungicide such as benomyl. Researchers have identified the fungus F. lateritium as a BCA against Eutypa lata (Munkvold, et al., 1993). Carter (1983) and Carter, et al. (1974 and 1975) describe the use of F. lateritium as a biocontrol agent for E. armeniacae (a relative of E. lata infecting apricot). F. lateritium and E. armeniacae were reported to have differential sensitivities to benomyl, of a magnitude of approximately 10 fold. In Carter, et al. (1975) apricot pruning wounds were treated with F. lateritium spores suspended in a solution of 125 ppm (approximately 2 μg/ml) benomyl, for control of E. armeniacae. In apple, F. lateritium and other fungi have been used to inhibit fungal pathogens on cankers (Grabowski, M., (1999); and Grabowski et al., (1997)).

Although F. lateritium has been identified as a BCA, its adoption for the control of E. lata has been limited due to performance inconsistency in the field. (Gendloff, et al., 1983). The natural resistance to benomyl in F. lateritium is very low, and so the advantage of applying F. lateritium as a BCA in an integrated management system with a chemical fungicide is limited.

Hence, there remains a need in the art for a biological control agent(s), formulations and composition comprising such agent(s) having resistance to very high benomyl concentrations, on the order of 1000 μg/ml of benomyl or more, and which can be used in methods for integrated biocontrol of plant pathogens by application directly to the plant at exposed wounds.

### RELEVANT LITERATURE

Carter, M. V. (1983). “Biological control of Eutypa armeniacae. 5. Guidelines for establishing routine wound protection in commercial apricot orchards.” Aust. J. Exp. Agric. Anim. Husb. 23: 429-436.

Carter, et al., (1974). “Biological Control of Eutypa armeniacae. II Studies of the Interaction between E. armeniacae and F. lateritium, and their relative Sensitiviies to Benzimidazole Chemicals”, Aust. J agric. Res., 1974, 25, 105-119.

Carter, et al., (1975). “Biological Control of Eutypa armeniacae. III A Comparison of Chemical, Biological and Integrated Control”, Aust. J agric. Res., 1975, 26, 537-5434.

Chet, et al., U.S. Pat. No. 4,915,944,“Novel isolate of Trichoderma, fungicidal compositions containing said isolate and use thereof”.

Gendloff, et al., (1983). “Fungicidal control of Eutpa armeniacae infection concord grapevine in Michigan.” Plant Disease 67(7): 754-756.

Gold, et al., (1991). Characterization of two beta-tubulin genes from Geotrichum candidum. Molecular and General Genetics 230(1-2): 104-112.

Grabowski, M., (1999). Folia-Horticulturae, 11(1): 29-35.

Grabowski et al., (1997). Phytopathologia-Polonica, 14:75-81.

Irelan, et al.; “Efficacy testing of Eutypa. Chemical and biological control candidates with DNA-based diagnostics”. Winegrowing (1999) January/February: 47-56.

Koenraadt, et al., (1992). Characterization of the beta-tubulin gene of benomyl resistant field strains of Venturia-inequalis and other plant pathogenic fungi. Phytopathology 82(11): 1348-1354.

McBeath, Jenifer H, U.S. Pat. No. 5,418,165, “Cold tolerant Trichoderma”.

Molnar, et al., (1985). The high level of Benomyl tolerance in Fusarium oxysporum is determined by the synergistic interaction of two genes. Experimental Mycology 9: 326-333.

Munkvold, et al., (1993). Efficacy of natural epiphytes and colonizers of grapevine pruning wounds for biological control of Eutypa dieback. Phytopathology 83(6): 624-629.

Orbach, et al., (1986). Cloning and characterization of the gene for beta-tubulin from a benomyl-resistant mutant of Neurospora crassa and its use as a dominant selectable marker. Molecular and Cellular Biology 6: 2452-2461.

Ossana, et al., “Genetic Transformation of the Biocontrol Fungus Gliocladium virens to Benomyol Resistance”, Appl. Envir. Microbiol., 1990, 56, 3052-3056.

Papavizas, George C., U.S. Pat. No. 4,489,161, “Strain of Trichoderma viride to control fusarium wilt”.

U.S. Congress, Office of Technology Assessment. 1995. Biologically Based Technologies for Pest Control. Washington, D.C, U.S. Government Printing Office.

Yan, et al., (1993). Sensitivity of field strains of Gibberella fujikuroi (Fusarium section liseola) to benomyl and hygromycin B. Mycologia 85(2): 206-213.

Yan, et al., (1996). Isolation of a β-Tubulin gene from Fusarium moniliforme that confers cold-sensitive Benomyl resistance. Applied and Environmental Microbiology 62(8): 3053-3056.

### SUMMARY OF THE INVENTION

A first aspect of the present invention relates to methods for treating a plant having a wound to increase resistance against a plant pathogen. The method involves applying an effective amount of a benomyl solution, in conjunction with inoculating the site of a wound on a plant with an antagonistic fungal organism resistant to said benomyl concentration. In a preferred embodiment, the biological control organism is F. lateritium.

The wound may arise from any number of causes. It may be a wound from physical damage, either natural or the result of human activity, such pruning or incident to orchard management. The wound may also be the result of pest damage, such as insect boring, or fungal or bacterial cankers.

In one preferred embodiment the wound is a pruning wound on grapevine, and the pathogen is E. lata. The invention also provides methods for utilizing such a strain for integrated biocontrol of the grapevine pathogen E. lata.

The benomyl concentration at which the fungal organism is resistant is preferably greater than about 10 μg/ml, and more preferably greater than about 100 μg/ml. Most preferably, the benomyl concentration is greater than about 1000 μg/ml.

Reports of benomyl resistant Fusarium have all involved pathogenic species. By this invention a resistant variety of a beneficial species, F. lateritium is provided as a biological control agent for an integrated disease management program.

In one embodiment the cells of F. lateritium are suspended in the benomyl solution, though they may be beneficially applied immediately before or after the application of benomyl. In this fashion the invention further provides a biological control composition for applying to the wound site of a plant which is a mixture of at least one fungal organism which is an antagonist against a plant fungal pathogen, and benomyl at a concentration of greater than about 2 μg/ml. The composition may include additives known to the art, selected from the group consisting of preservatives, carriers, surfactants, wetting agents and mixtures thereof.

The methods and composition may be used in treating any wounded site on a plant which may provide fungal pathogens with an avenue of infection on the plant. Plants where the invention may be used include grapevine, apricot, citrus, apple, pear, persimmon, peach, cherry and plum. A preferred embodiment involves applying the compositions immediately upon pruning of a plant, most preferably at every, or nearly every, pruning wound so created.

In a preferred embodiment the cells of Fusarium are applied as conidial spores or chlamydospores at concentrations of about 10⁵, and up to about 10⁸ spores per ml.

A second aspect of the present invention relates to a strain of a fungal organism which is antagonistic to a fungal pathogen at a site of pruning wounds. The invention provides a biologically pure culture of an isolate of F. lateritium having the identifying characteristics of isolate AJS101 (ATCC Deposit No. ______, Mar. 2, 2001).

In regard to the second aspect of the invention, strains of F. lateritium that are highly resistant to benomyl are provided. The development of a strain of benomyl resistant F. lateritium was performed through mutagenesis and selection. The mutant strain shows resistance to very high benomyl concentrations, and can grow at 1000 μg/ml of benomyl, whereas the wild type has a natural low resistance to concentrations of about 2 μg/ml benomyl.

In a preferred embodiment of the invention, wound sites are subjected to an aqueous suspension comprising cells of the fungal organism antagonistic to the fungal plant pathogen, and also subject to an application of the fungicide, either simultaneously or immediately before or after. In a preferred embodiment the antagonistic fungal organism is F. lateritium.

### BRIEF DESCRIPTION OF THE DRAWINGS

FIG. 1. Mean colony diameter measurements taken after a three-day incubation period for WT F. lateritium. Growth was not observed at benomyl concentrations greater than 1 μg/ml. At 4° C. growth was not observed at 1 μg/ml benomyl.

FIG. 2. Mean colony diameter measurements taken after a three-day incubation period for BR. F. lateritium. Growth was not observed at 4° C. when the benomyl concentration exceeded 10 μg/ml and at 29° C. when the concentration exceeded 100 μg/ml.

FIG. 3.(a) Fermentation characteristics of the WT and BR F. lateritium strains produced in potato dextrose broth. (b) E. lata control levels for bioassays conducted using WT and BR F. lateritium fermentation samples at 10° C. All bioassays were performed in triplicate.

### DETAILED DESCRIPTION OF THE INVENTION

Aspects of the present invention may be practiced with a variety of beneficial fungal organisms which are antagonistic against plant fungal pathogens. As used herein, an “antagonistic organism”, or an “antagonist to a fungal organism” means any fungal organism which will inhibit the growth of a fungal pathogen. Antagonistic organisms useable in the present invention may, for example, be identified by following the procedure of screening fungal isolates from the field (e.g. taking isolates from exposed surface(s) of plants, such as pruning wounds), and isolating such fungal organism(s). These organisms are then screened for antagonistic effect against plant pathogens. Such fungal organisms may, for example, exert their antagonism by either out-competing the pathogen for available nutrients or rendering the infection site unfavorable for the pathogen, such as by production of antagonistic substances. They are also capable of colonizing the exposed areas of a pruned plant, such as at a pruning wound.

Fusarium is one example of fungal genera that produce mycotoxins, anti-fungal compounds, and hence can function as antagonistic fungal organisms. The biological control of E. lata by F. lateritium may arise from the action of a mycotoxin, and/or from a competitive growth advantage on the pruning wound (i.e., the organisms multiply and occupy the surfaces of wound, thereby excluding infection by pathogens). The strain of benomyl resistant F. lateritium designated AJS101 is deposited with the American Type Culture Collection under accession number ______.

As used herein, the term “benomyl or benomyl-like pesticide” refers to 1-(butylcarbomyl)-2-benzimidazole carbamate or a compound that is a homolog of Benomyl or exhibits substantially the same functional properties as Benomyl.

A “BR strain” of F. lateritium, as used herein, means a strain which is capable of growing on benomyl at concentrations of greater than about 2 μg/ml. A preferred BR strain of F. lateritium will grow at greater than about 10 μg/ml, and most preferably from about 100 μg/ml up to about 1000 μg/ml or greater benomyl. Benomyl-resistant (BR) and wild-type (WT) strains of F. lateritium both show an antagonistic effect to E. lata. The BR strains will find particular use in integrated pest or disease management programs, as the biological control agent (or BCA).

In order to isolate a benomyl resistant strain of F. lateritium, several attempts were made to find a natural mutant in the wild-type population. WT F. lateritium was streaked onto potato dextrose agar plates containing 1200 μg/ml of benomyl, and the plates were incubated at 28° C. for 3 to 4 days. No growth was observed in this study and two subsequent studies. Benomyl concentrations were lowered to 300, 120, 60, and 12 μg/ml, but growth was still not observed on these plates. Higher inoculum produced by centrifuging 1 ml of four-day-old flask growths was also not successful in producing a natural mutant.

UV mutagenesis was then performed on four day old cultures of F. lateritium after spreading approximately 1×10⁷ conidia from 1 ml of centrifuged cells on PDA plates containing 0, 12, or 60 μg/ml benomyl. Growth appeared on plates containing 0 or 12 μg/ml benomyl, but not on the 60 μg/ml plates. Strains were transferred to other plates containing benomyl to confirm the resistance, and for storage.

Fungal organisms may be tested for resistance to fungicides, (e.g. growing the fungal organisms on media comprising a fungicide, i.e., a benzimidazole fungicide) either before or after mutagenesis. In a preferred method, screening is accomplished by first screening the fungal organism on media having a relatively low concentration of fungicide, i.e., from about 100% to 1000% of the normal tolerance level of the wild type for said fungal organism. Fungal organisms growing at this level are then transferred to progressively higher concentrations of the fungicide.

The BR strain of F. lateritium provided by this invention is able to grow in the presence of benomyl. Both the WT and BR strains are effective in the control of E. lata. Differences in the growth characteristics of the WT and BR strains were assessed, and the effect of fermentation culture age on efficacy. Because benomyl is the preferred means of control of E. lata, dosage response studies on both the WT and the BR strain of F. lateritium were also conducted.

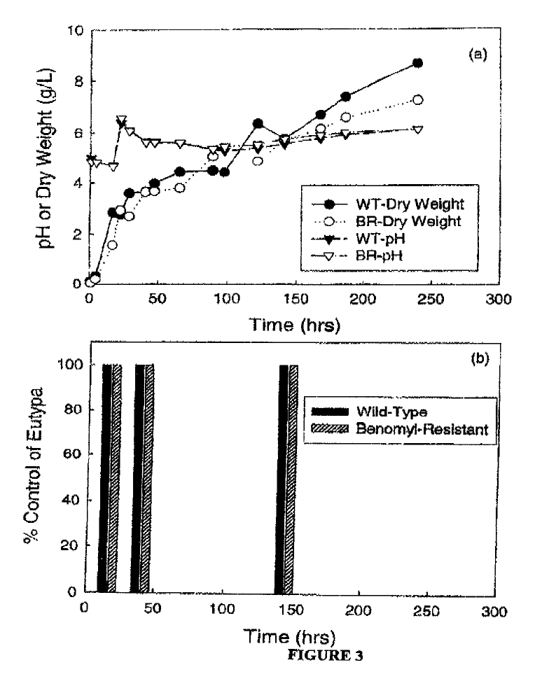

After determining that the isolated strain was benomyl resistant, the fermentation characteristics and efficacy of the newly isolated strains were examined. Parallel 4.5-L (working volume) fermentations using PDB for medium were inoculated in instrumented fermentors, one with the WT strain and one with the BR strain. As illustrated in FIG. 3(a), fermentation characteristics such as pH and cell concentration for the two strains were nearly identical. Biological assays were also performed on samples from these two fermentations. Results from the bioassays incubated at 10° C. are presented in FIG. 3(b). Both WT and BR F. lateritium fermentation samples from 16, 40, 144, and 240 hours into the fermentation were effective in controlling E. lata. Similar results were obtained in bioassays incubated at 26° C. In all cases, full control of E. lata was observed for both the WT and BR strains of F. lateritium, indicating that the newly-isolated strain possesses at least comparable efficacy.

Samples taken from the fermentors confirmed that the BR strain isolated from the end of the fermentation remained benomyl-resistant.

The BR and WT strains of F. lateritium were examined for their tolerance to benomyl on potato dextrose agar (PDA) containing benomyl and control of Eutypa lata in grapevine bioassays. The WT strain grew on PDA containing 1 μg/ml benomyl at 13° C., 26° C. and 29° C. The BR strain grew on PDA containing 10 μg/ml benomyl at 4° C., on PDA containing 100 μg/ml benomyl at 29° C., and on PDA containing 1000 μg/ml benomyl at 13° C. and 26° C. The BR strain was also able to colonize grapevine segments and control E. lata in the presence of 1000 μg/ml benomyl.

The WT F. lateritium strain was moderately tolerant to benomyl. This level of tolerance to benomyl has been observed in other fungi (Molnar, et al., 1985; Yan, et al., 1993; and Yan, et al., 1996). Both the WT and BR F. lateritium strains displayed some level of cold sensitivity when grown on PDA containing benomyl. The WT strain grew on PDA containing 1 μg/ml benomyl at 13° C., 26° C. and 29° C., but did not grow at 4° C. Likewise the BR strain grew on PDA containing 100 μg/ml benomyl at 13° C., 26° C. and 29° C., but did not grow at 4° C. Cold sensitivity to benomyl is not uncommon for benomyl-resistant fungi (Yan, et al., 1996). Cold sensitivity must be considered since the temperature during grapevine pruning, and thus the window for E. lata infection, ranges between 5° C. and 25° C. Because of the variance in temperature during the window of infection and the observed cold sensitivity of both WT and BR strains, efforts are now under way to determine the behavior of the WT and BR F. lateritium under the temperature range of 5-15° C.

At 26° C. and 29° C. colony diameters were greater for the WT F. lateritium when grown on PDA containing 1 μg/ml benomyl than with no benomyl. Furthermore, on PDA containing no benomyl WT F. lateritium colony diameters were smaller at 26° C. and 29° C. than at 13° C. Benomyl and temperature levels may have affected the biomass density of the colonies, and subsequently, the colony diameters. Since colony densities were not measured in this study, it is difficult to conclude whether or not WT F. lateritium colonized PDA plates better in the presence or absence of 1 μg/ml benomyl at 13° C., 26° C. and 29° C.

The present suggested field application concentration of benomyl for the control of E. lata in grapevine is 12,000 μg/ml (1.6 oz/gal). The rates in studies presented in this paper ranged from 0 to 1000 μg/ml, with sensitivity at 10,000 μg/ml. Although the benomyl tolerance levels of the BR strain were lower than the benomyl application levels presently employed in the field, the observed tolerance levels were still quite high, and may exceed the level of 1000 μg/ml. If the benomyl application concentrations in the field are lowered below the suggested level to near 1000 μg/ml, the BR strain could be applied concurrently in the field with benomyl. This provides both short term and long term control of E. lata.

The longer term control provided by the antagonist fungal organism may permit the use of fewer applications of benomyl, or even the use of lower concentrations of benomyl, as part of an integrated disease management program. With the lower concentrations (1000 μg/ml and less) the formulations become less viscous, providing a further advantage in ease and options in application of benomyl in the field.

The fermentation results demonstrate that both the WT and BR strains of F. lateritium are amenable to production via liquid fermentation. Developing the other parameters related to the development of the fungi as BCAs, such as shelf life, storage and handling, are well within the skill of the art. Fusarium produces three kinds of spores: macroconidia, microconidia and chlamydospores. Macro- and microconidia are dispersed during rain, which act as continual inoculum. Chlamydospores persist in the soil for many years. Chlamydospores are round, one- or two-celled, thick-walled spores produced terminally or intercalary on older mycelium. Any of these forms may be utilized, or mixtures thereof, in the compositions and methods of this invention.

There were no observed differences in the efficacy of strains of F. lateritum against E. lata. Both the BR and WT strains controlled E. lata at 10 and 26° C. at several stages of growth. Although control of E. lata was achieved with WT and BR F. lateritium under a variety of conditions, the results from the bioassays in this study are inconclusive as to the exact mechanism of biocontrol. Further research is needed in this particular area as it would greatly aid the development of production methods for F. lateritium as a BCA.

Both strains were amenable to production via liquid fermentation and both achieved 100% control of E. lata in grapevine bioassays. Neither the duration of fermentation nor incubation temperature during grapevine bioassays influenced the efficacy of either strain against E. lata. The results show that application of BR F. lateritium alone or in combination with benomyl can be used to provide good control of E. lata.

The methods utilizing the BR strain of F. lateritium of the invention will find use in controlling plant pathogens on a variety of agricultural tree and vine crops which are susceptible to die-back or fungal canker diseases, including, but not limited to grapevine, nut crops, citrus crops, stone fruits, apples, pears and persimmons.

Citrus fruit may include, for example, grapefruit, orange, lemon, kumquat, lime and pummelo. Nuts may, for example, include almonds and pecans. Stone fruits may include, for example, apricot, peach, cherry and plum.

When grown in a liquid medium, the fungal organisms may be applied in suspension with the liquid medium, however it is preferred in order to improve control of application, to apply the fungal organisms in the presence of or one or more of carriers. Compositions of the present invention may also include other additives including pesticides, such as fungicides (e.g. “Benlate®” available from DuPont). If applying the fungal organism in conjunction with a fungicide, the composition will be formulated in accordance with the directions for application of the fungicide.

Other additives know to the art may include additives which promote spreading of the compositions of the present invention, additives which promote sticking of the compositions of the present invention to the pruning wounds, nutrients for the fungal cells of the present invention, and mixtures of the aforementioned additives. When used, these additives should be used in an amount(s) which will not interfere with the growth, development or effectiveness of the fungal organism(s) of the present invention. Typically, preparation of suitable compositions require only mixing of the fungal organism(s) with the additives. Typical preparation includes, adding together the fungal organism(s), preservative and powdered ingredient, and then mixing and/or grinding the constituents together. The powdered composition may be dusted on an agricultural commodity, or the powdered composition may be mixed with liquid (e.g. water) and subsequently applied to an agricultural commodity. The compositions of the present invention will have excellent storage properties, will not require refrigeration, will not typically encounter contamination problems, and will remain effective in typical agricultural storage environments.

When the compositions of the present invention are in the form of a liquid mixture or suspension, any concentration of constituents may be used which inhibits plant pathogen development of the targeted plant pathogen when applied to a the wound site. As will be apparent to one skilled in the art, effective concentrations may vary depending upon such factors as: (1) the type of plant; (2) the physiological condition of the plant (e.g. the stage of the growing season); (3) the concentration of pathogens affecting the plant; (4) the type of wound on the plant; (5) temperature and humidity; and (6) the stage of the plant pathogen. Exemplary concentrations range from about 10⁵ up to about 10⁸ CFU/ml of liquid composition. The optimal concentration for any application can be easily determined by the skilled artisan through known testing procedures.

The fungal organism(s) of the invention may be applied to plants using conventional methods such as dusting, spraying or brushing. In addition, the microorganisms of the invention may be incorporated into a variety of compositions suitable for application to the plants.

Bioassays and colonization studies were completed on grapevine segments to confirm the control of E. lata by the BR F. lateritium and the ability of both organisms to colonize grapevine segments in the presence of benomyl. Results from these studies are listed in Table 1. The BR F. lateritium controlled E. lata and colonized grapevine segments for each benomyl concentration tested. E. lata did not colonize segments when the benomyl concentration applied to the segments exceeded 100 μg/ml.

TABLE 1
Colonization of grapevine segments and control
of E. lata by the BR F. lateritium in the presence of benomyl.

| Benomyl | | E. lata | F. lateritium |
|---|---|---|---|
| conc. | Treatment | colonization | colonization |
| (μg/ml) | (number per treatment) | (%) | (%) |
| 0 | F. lateritum (12) | 0 | 100 |
| | E. lata (12) | 100 | 0 |
| | F. lateritium & E. lata (3) | 0 | 100 |
| 10 | F. lateritum (3) | 0 | 100 |
| | E. lata (3) | 100 | 0 |
| | F. lateritium & E. lata (3) | 0 | 100 |
| 100 | F. lateritum (3) | 0 | 100 |
| | E. lata (3) | 90 | 0 |
| | F. lateritium & E. lata (3) | 0 | 100 |
| 1000 | F. lateritum (3) | 0 | 100 |
| | E. lata (3) | 0 | 0 |
| | F. lateritium & E. lata (3) | 0 | 100 |

### EXAMPLES

### Example 1

Isolation of the Benomyl-Resistant F. lateritium Strain

After random mutagenesis and selection the BR F. lateritium strain was isolated. First, potato dextrose agar (Difco) plates (PDA) were prepared using 0, 12, and 60 μg/ml of benomyl. Benomyl was added as a powder to the PDA after autoclaving PDA and prior to pouring. F. lateritium was grown for four days in 100 ml of potato dextrose broth (PDB) in a 500 ml flask shaken at 220 rpm at room temperature. One-ml samples of cells were removed and centrifuged in sterile 1.5-ml microcentrifuge tubes. The supernatant was discarded and the cell pellets aseptically transferred to four of each type of benomyl plate. The first plate of each benomyl concentration was exposed to two 15-watt UV light sources for two minutes, the second plate for one minute, and the third plate for 30 seconds. The remaining plates were left as controls. The UV light source was positioned 60 cm above each plate. After UV exposure, the plates were wrapped with aluminum foil and incubated for three to four days at 28° C. Growth on the various plates was noted and restreaked onto PDA plates of 0, 12, and 60 μg/ml benomyl. These plates were also incubated at 28° C. for three to four days. Benomyl resistance was maintained during repeated plate-to-plate transfers.

### Example 2

Culture Maintenance

Both WT and BR F. lateritium strains were maintained in shake flask cultures containing PDB at 30° C. and 150 rpm, and on PDA at 26° C. The BR F. lateritium strain was also maintained on PDA containing 100 μg/ml benomyl at 26° C.

### Example 3

Benomyl Tolerance Assays on PDA

In this study, tolerance to benomyl is considered to mean the ability to grow in the presence of 10 μg/ml benomyl or more. Tolerance assays were completed in 15-cm diameter petri dishes containing different levels of benomyl in PDA. Media were prepared aseptically by adding benomyl suspended in 10 ml of acetone to 990 ml of autoclaved PDA cooled to 60° C. The benomyl concentrations tested were 0, 1, 10, 100, and 1000 μg/ml benomyl. Media were set overnight at 4° C. prior to inoculation.

Inoculation was completed using 21 μl of three-day-old shake flask cultures of BR and WT F. lateritium in three 7-μl aliquots. Each benomyl concentration was tested twice. Once inoculated, the plates were placed in incubators for three days. The incubation temperatures studied were 4° C., 13° C., 26° C. and 29° C. Colony diameter was measured after the three-day incubation period using a computer imaging system (Alpha Innotech Corp., San Leandro, Calif.). Mean colony diameters and standard errors about the mean were calculated for each treatment.

Benomyl Tolerance of WT and BR F. lateritium on PDA Containing Benomyl

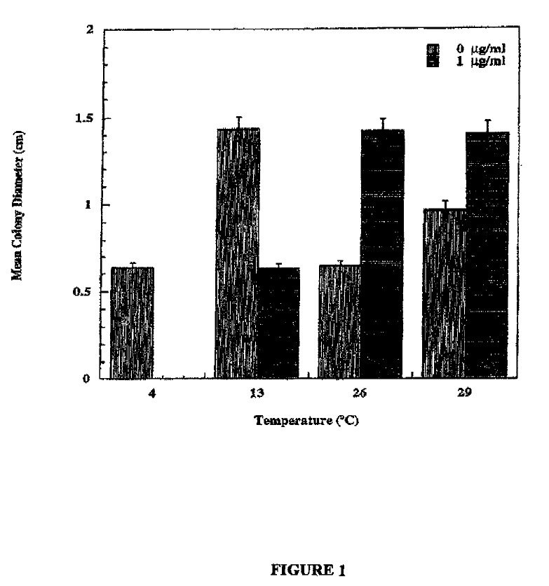

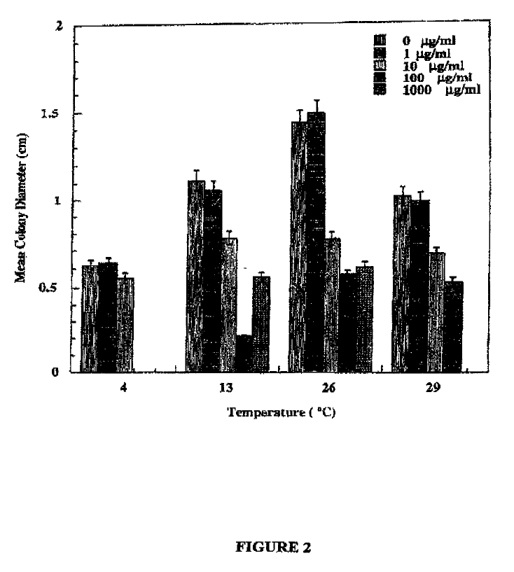

Colony diameter data for WT and BR F. lateritium strains after incubation for three days on PDA plates containing benomyl is illustrated in FIGS. 1 and 2. Wild-type F. lateritium did not grow at benomyl levels above 1 μg/ml (data not shown). At 4° C., no growth of WT F. lateritium was observed in the presence of 1 μg/ml benomyl. When grown on PDA containing no benomyl, the WT F. lateritium mean colony diameters increased as temperature increased from 4° C. to 13° C., but decreased as temperature increased above 13° C. When WT F. lateritium was grown on PDA containing 1 μg/ml benomyl, mean colony diameters were greatest at 26° C. and 29° C.

The BR F. lateritium strain was capable of growing in the presence of high concentrations of benomyl over a range of temperatures (FIG. 2). However, the BR strain did not grow at benomyl concentrations of 100 μg/ml or greater when the incubation temperature was 4° C. Growth was also inhibited on PDA containing 1000 μg/ml when the incubation temperature was 29° C. Mean colony diameters for the BR strain were greatest at 26° C. for almost all benomyl concentrations tested. For experiments conducted at 13° C., 26° C., and 29° C., colony diameters tended to decrease as the concentration of benomyl increased. There was a slight decrease in colony diameter at 4° C. as the benomyl concentration increased from 0 to 10 μg/ml.

### Example 4

Fermentation Studies

Fermentation studies were carried out using both the WT and BR strains of F. lateritium. Inoculum for each fermentation was prepared aseptically by transferring a 1-2 cm² colonized piece of maintenance PDA culture into 100 ml of sterile PDB in a 500 ml shake flask. The shake flasks were incubated for approximately 4 days at 220 rpm at 27° C.

Shake-flask cultures were used to inoculate 4.5 L of sterile PDB in a fully-instrumented Bioflow 3000 fermentor (New Brunswick Scientific, New Brunswick, N.J.). Dissolved oxygen and pH were measured online using sterilized dissolved oxygen probes and pH electrodes. Agitation rate was maintained at 300 rpm and aeration was maintained at 4.5 L/min. Samples were removed daily for analysis of cell morphology, biomass concentration, carbohydrates and nitrogen (both ammonia and primary amino nitrogen). Biomass concentration was monitored using 10-ml samples of fermentation both. Samples were filtered through a preweighed filter, washed with 10 ml of water, and then dried in a microwave oven to constant weight (approximately 4 min). Fermentations were also sampled at 16, 40, 144, and 240 hours after inoculation for biological efficacy analyses. Efficacy was evaluated using the bioassay described below.

### Example 5

Grapevine Bioassays

Bioassays were conducted in a manner similar to that described by Munkvold, et al. Bioassays used 4 to 5 day-old cultures of E. lata produced in PDB shake-flask culture at 30° C. and 150 rpm. These cultures yielded approximately 1×10⁵ cells/ml. 1.5-cm segments of 1-year old Zinfandel grapevine canes were used in all bioassays. Canes were obtained from the UC Davis campus vineyard and stored at −20° C. until needed. Bark was removed from the segments, before they were autoclaved for 30 minutes. The autoclaved segments were then embedded upright in water agar in 15-cm diameter petri dishes (5 segments per dish). First, segments were inoculated with 25 μl of broth from the F. lateritium fermentations. Two days after the inoculation of F. lateritium, segments were inoculated with 25 μl of broth from the E. lata shake-flask cultures and then incubated for 10 days at either 26 or 10° C.

Bioassays were also completed to evaluate the tolerance of the BR F. lateritium strain and E. lata to benomyl when applied to grapevine segments. Benomyl concentrations tested included 0, 10, 100 and 1000 μg/ml at 50 μl aliquots per segment. Bioassays were inoculated with 20 μl aliquots of broth from 4 to 5 day old shake flask cultures of BR F. lateritium and E. lata. The F. lateritium shake flask cultures yielded approximately 4.5×10⁵ cells/ml. Segments were inoculated first with benomyl and then with E. lata and BR F. lateritium. All inoculations were completed within one hour in a laminar flow hood. These bioassays were incubated at 26° C. for 10 days.

After incubation, segments were removed from the water agar, split in half and surface disinfected for ½ hour in a 2.5% sodium hypochlorite solution. The split segments were plated on PDA and monitored for E. lata presence after 5 days. All bioassays were performed in triplicate. Positive and negative controls were performed for each bioassay. Mean percent infection levels and standard errors about the mean were calculated for each treatment.

Although the foregoing invention has been described in some detail by way of illustration and example for purposes of clarity of understanding, it will be obvious that certain changes and modifications may be practiced within the scope of the appended claim.

What is claimed is:

1. A method for treating a plant to increase resistance against a plant pathogen, said method comprising the steps of:
a) applying to a wound of said plant a benomyl solution having a concentration of greater than about 2 μg/ml benomyl, and
b) inoculating said wound with cells of a fungal organism which is an antagonist to said plant pathogen,
wherein said fungal organism is resistant to said benomyl concentration.

2. The method of claim 1, wherein said wound is pruning wound.

3. The method of claim 1, wherein said benomyl concentration is greater than about 10 μg/ml.

4. The method of claim 3, wherein said benomyl concentration is greater than about 100 μg/ml.

5. The method of claim 4, wherein said benomyl concentration is greater than about 1000 μg/ml.

6. The method of claim 1, wherein said cells are suspended in said benomyl solution.

7. The method of claim 1, wherein said plant is a grapevine.

8. The method of claim 1, wherein said fungal organism is F. lateritium.

9. In a method for treating a plant for resistant against pathogens, said method comprising applying benomyl to an exposed wound of said plant in a solution having a concentration of greater than about 2 μg/ml benomyl,
the improvement comprising adding to said solution cells of a fungal organism is resistant to said benomyl concentration.

10. The method of claim 9, wherein said plant is a grapevine.

11. The method of claim 9, wherein said benomyl concentration is greater than about 10 μg/ml.

12. The method of claim 11, wherein said benomyl concentration is greater than about 100 μg/ml.

13. The method of claim 12, wherein said benomyl concentration is greater than about 1000 μg/ml.

14. The method of claim 9 wherein said fungal organism is F. lateritium.

15. The method of claim 14, wherein said F. lateritium cells are conidial spores.

16. The method of claim 14 wherein said F. lateritium cells are chlamydospores.

17. A strain of F. lateritium tolerant to a concentration of greater than about 2 μg/ml benomyl.

18. The F. lateritium strain of claim 17, tolerant to greater than about 10 μg/ml benomyl.

19. The F. lateritium strain of claim 18, tolerant to greater than about 100 μg/ml benomyl.

20. The F. lateritium strain of claim 19, tolerant to greater than about 1000 μg/ml benomyl.

21. The F. lateritium strain of claim 17, wherein said strain is ______(ATCC Deposit No. ______).

22. A biological control composition for application to a wounded site of a plant, said composition comprising cells of at least one fungal organism capable of growth at a benomyl concentration of greater than about 2 μg/ml, and which is an antagonist to a plant fungal pathogen,
and a dispersal medium for applying said composition to said wounded site.

23. The composition of claim 22 further including an additive selected from the group consisting of preservatives, carriers, surfactants, wetting agents and mixtures thereof.

24. The composition of claim 22 wherein said plant is selected from the group consisting of grapevine, stone fruits, nut trees, apple, pear, persimmon and citrus.

25. The composition of claim 22 wherein said stonefruit is selected from the group consisting of apricot, peach, cherry and plum.

26. The composition of claim 22, wherein said fungal organism is F. lateritium.

27. The composition of claim 22, wherein said cells have a concentration of at least about 10⁵ spores per gram of said composition.

28. The composition of claim 27, wherein said cells have a concentration of at least about 10⁶ spores per gram of said composition.

29. The composition of claim 28, wherein said cells have a concentration of at least about 10⁷ spores per gram of said composition.

30. The composition of claim 29, wherein said cells have a concentration of at least about 10⁸ spores per gram of said composition.

31. The composition of claim 22 wherein said cells are selected from the group consisting of conidial spores, chlamydospores and a mixture of conidial spores with chlamydospores.

32. The composition of claim 22 further comprising benomyl at a concentration of greater than about 2 μg/ml benomyl.

33. The composition of claim 32 wherein said benomyl concentration is greater than about 10 μg/ml benomyl.

34. The composition of claim 33 wherein said benomyl concentration is greater than about 100 μg/ml benomyl.

35. The composition of claim 34 wherein said benomyl concentration is greater than about 1000 μg/ml benomyl.

36. A method for obtaining a beneficial fungal organism resistant to benomyl, said method comprising the steps of:
a) exposing cells of said organism to a mutagen;
b) growing mutated cells of said organism on a culture medium comprising a low benomyl concentration of less than about 50 μg/ml benomyl;
c) selecting mutated cells capable of growing on said medium;
d) transferring said mutated cells onto culture medium onto culture medium having a high benomyl concentration of greater than about 50 μg/ml benomyl; and
e) selecting benomyl resistant strains capable of growing on said high benomylo concentration mudium.

37. The method of claim 36, further comprising in step d) transferring said cells onto progressively higher concentrations of benomyl culture medium.
